(19) United States
(12) Plant Patent Application Publication
(10) Pub. No.: US 2004/0098777 P1
(43) Pub. Date: May 20, 2004

(54) Lomandra longifolia plant named 'LM400'

(76) Inventor: Todd Anthony Layt, New South Wales (AU)

Correspondence Address: MYERS BIGEL SIBLEY & SAJOVEC, PO BOX 37428, RALEIGH, NC, 27627, US

(21) Appl. No.: 10/626,680

(22) Filed: Jul. 24, 2003

Related U.S. Application Data
(60) Provisional application No. 60/400,389, filed on Jul. 31, 2002.

(51) Int. Cl. A01H 5/00

(52) U.S. Cl. PLT/263

(57) ABSTRACT

‘LM400’ is a distinctive variety of Lomandra longifolia which is characterized by the combination of its dwarf, compact growth habit; its fine, narrow leaf blade; and the blue/grey color of the leaf blade.

### RELATED APPLICATION INFORMATION

This application claims the benefit of U.S. Provisional Application Serial No. 60/400,389 filed 31 Jul. 2002, the disclosure of which is incorporated herein by reference in its entirety.

### LATIN NAME OF THE GENUS AND SPECIES

The Latin name of the genus and species of the novel variety disclosed herein is Lomandra longifolia.

### VARIETY DENOMINATION

The inventive variety of Lomandra disclosed herein has been given the variety denomination ‘LM400’.

### BACKGROUND OF THE INVENTION

The present invention relates to a new and distinct perennial grass variety of Lomandra longifolia, which has been given the variety denomination of ‘LM400’. Its market class is that of an ornamental grass. ‘LM400’ is intended for use in landscaping and as a decorative grass plant.

The Lomandra longifolia variety ‘LM400’ was first discovered in January, 1999 in an Australian nursery in the state of New South Wales during a routine inspection of large quantities of Lomandra longifolia ‘Cassica’ (Australian Plant Breeders' Rights Application No.1997/166) production stock. ‘LM400’ is believed to be a sport of ‘Cassica’. ‘LM400’ was selected due to its much finer leaf and compact size. ‘LM400’ was first propagated asexually by division in the state of New South Wales, Australia and has since been asexually propagated by division. The distinctive characteristics of the inventive ‘LM400’ variety are stable from generation to generation; clones of the variety produced by asexual reproduction maintain the distinguishing characteristics of the original plant.

‘LM400’ has a dwarf and compact growth habit with leaves that are finer and more narrow than Lomandra longifolia ‘Katrina’ (Australian Plant Breeders' Rights Application No. 1997/168), ‘Cassica’, and common Lomandra longifolia. It has a more blue/grey leaf color than Lomandra longifolia ‘LM300’ (U.S. Plant patent application Ser. No. 10/402,494; Australian Plant Breeders' Rights Application No. 2001/092).

An application for plant breeders' rights for variety ‘LM400’ has been lodged with the Australian Plant Breeders' Rights Office, and was first gazetted in August 2001 under Application No. 2001/090.

### SUMMARY OF THE INVENTION

‘LM400’ is a distinctive variety of Lomandra longifolia which is characterized by the combination of its dwarf, compact growth habit; its fine, narrow leaf blade; and the blue/grey color of the leaf blade.

### BRIEF DESCRIPTION OF THE FIGURES

FIG. 1 shows an ‘LM400’ plant.

FIG. 2 shows a comparison of the leaf width of ‘LM400’ with several known varieties of Lomandra longifolia plants, from left to right, common Lomandra longifolia, ‘Katrina’, ‘Cassica’, ‘LM400’ and ‘LM300’.

FIG. 3 shows a comparison of the leaf width of ‘LM400’ (left) and ‘LM300’ (right).

FIG. 4 shows a comparison between ‘LM300’ and ‘LM400’ (right) plants.

FIG. 5 provides a comparison of the length of the leaf of ‘LM400’ with several known varieties of Lomandra longifolia plants, from left to right, common Lomandra longifolia, ‘Katrina’, ‘Cassica’, ‘LM400’ and ‘LM300’.

FIG. 6 shows a comparison of the basal shoots of ‘LM400’ (left) and ‘LM300’ (right).

FIG. 7 shows another comparison between the basal shoots of ‘LM400’ (left) and ‘LM300’ (right).

FIG. 8 shows a comparison of the seed heads of ‘LM400’ with several known varieties of Lomandra longifolia, from left to right, ‘Katrina’, ‘Cassica’, ‘LM400’ and ‘LM300’.

### BOTANICAL DESCRIPTION OF THE PLANT

The following is a detailed botanical description of a new and distinct variety of a Lomandra longifolia ornamental grass known as ‘LM400’. The descriptions disclosed herein are based upon observations made in April/May 2002 of mature ‘LM400’ plants grown in nursery pots and field plots in New South Wales, Australia. Those skilled in the art will appreciate that certain characteristics will vary with older or, conversely, younger plants. ‘LM400’ has not been observed under all possible environmental conditions. Where dimensions, sizes, colors and other characteristics are given, it is to be understood that such characteristics are approximations or averages set forth as accurately as practicable. The phenotype of the variety may vary with variations in the environment such as season, temperature, light intensity, day length, cultural conditions and the like.

‘LM400’ is a perennial Lomandra longifolia plant which is believed to be a sport of ‘Cassica’. After its selection, ‘LM400’ was asexually propagated by division. ‘LM400’ is a narrow-bladed, compact plant, which is unusual for Lomandra longifolia plants which usually have a medium-to-wide blade and are larger than ‘LM400’ in most respects.

Growth Habit, Dimensions and Color

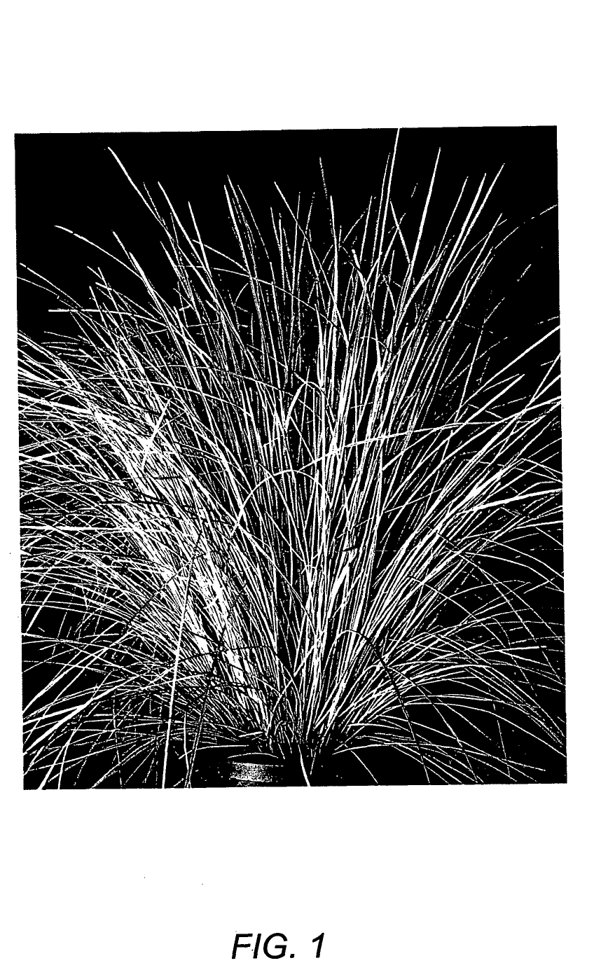

‘LM400’ is a very short, rhizomatous plant forming a compact tussock. The leaf is a blue/grey color. The leaf is generally 3 to 5 mm in width, which is unusual for this species, and reaches an average length of 714 mm. The basal shoots are white/green and light brown in color. The leaf is generally a uniform width from base to the tip, with a jagged tip typical of Lomandra longifolia. The inflorescence (flower spike) is yellow with an average length of 185 mm and an average width of 25 mm. To the touch, the flower spike is medium spiky, which is unusual for this species. A representative ‘LM400’ plant is shown in FIG. 1.

‘LM400’ has good color retention in winter compared with other varieties of Lomandra longifolia, with the exception of ‘LM300’ which has better winter color retention then ‘LM400’. These features and other characteristics are apparent from the figures provided herein.

Comparison of ‘LM400’ with Other Varieties of Lomandra longifolia

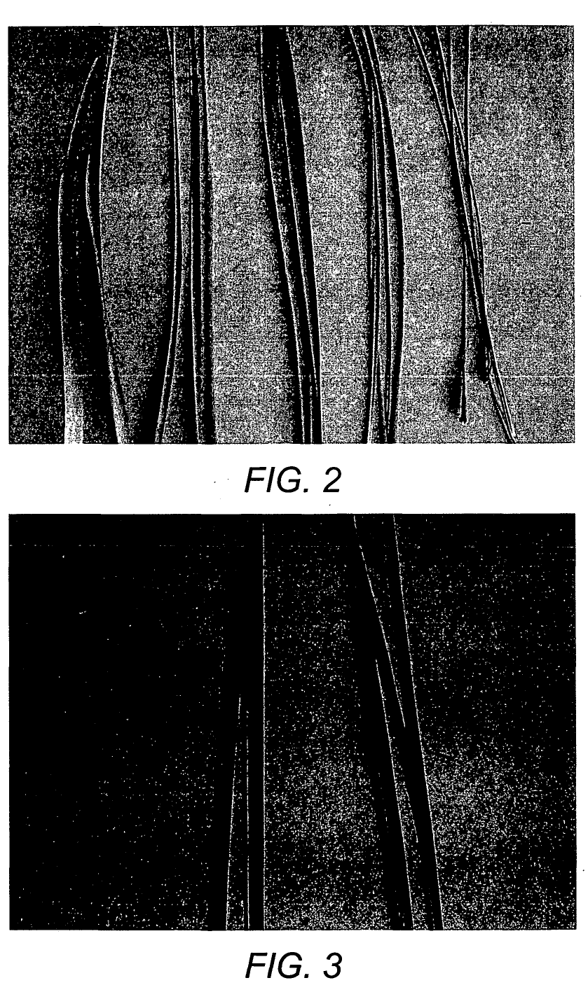

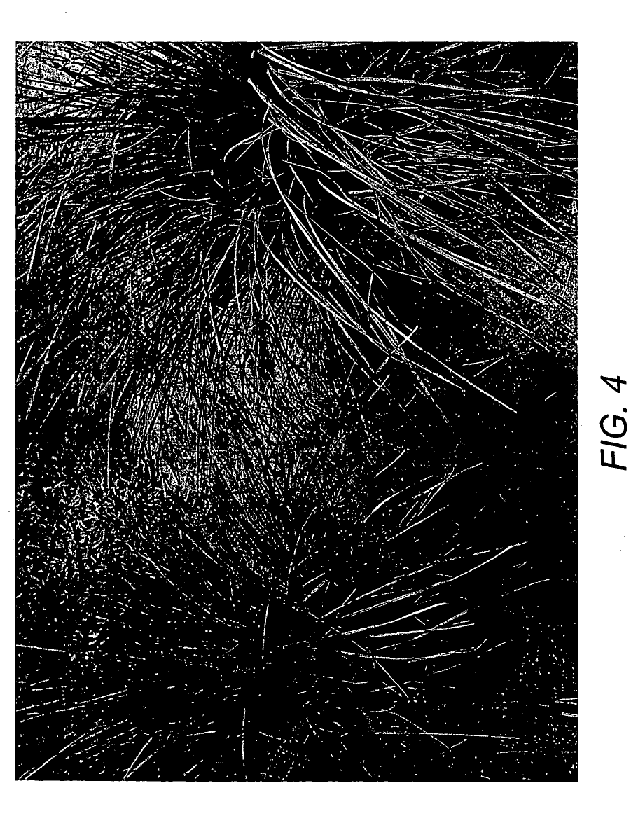

The leaf blade width of ‘LM400’ is finer and narrower than that of ‘Cassica’ and other comparable types of Lomandra longifolia (FIG. 2). The blade of ‘LM300’ is slightly more narrow than the blade of ‘LM400’ (FIG. 3 and FIG. 4).

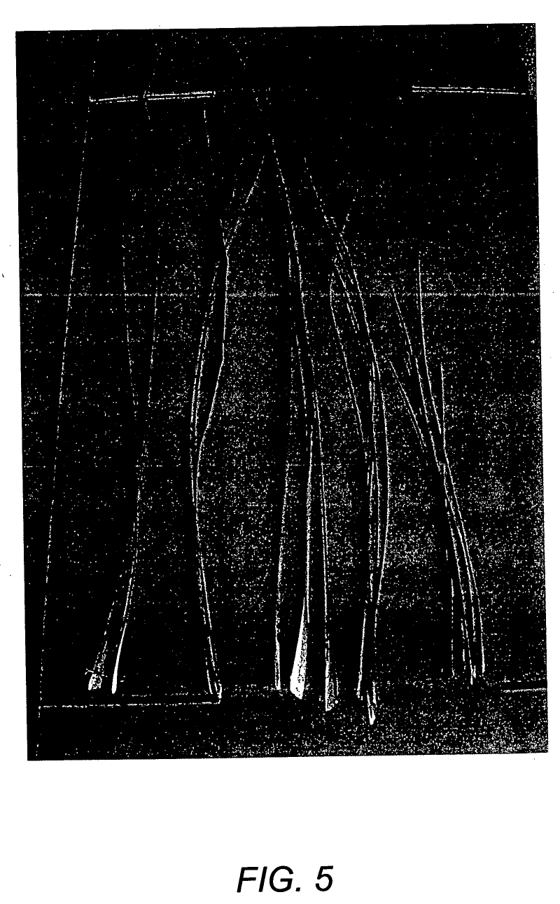

‘LM400’ also has a more compact habit than ‘Katrina’, ‘Cassica’ and other common forms of Lomandra longifolia (FIG. 5). ‘LM300’ is slightly more compact then LM400. The combination of its compact form and narrow leaf makes ‘LM400’ an attractive ornamental grass-like plant, unlike its parent, which is more characterized as a strappy leaf plant.

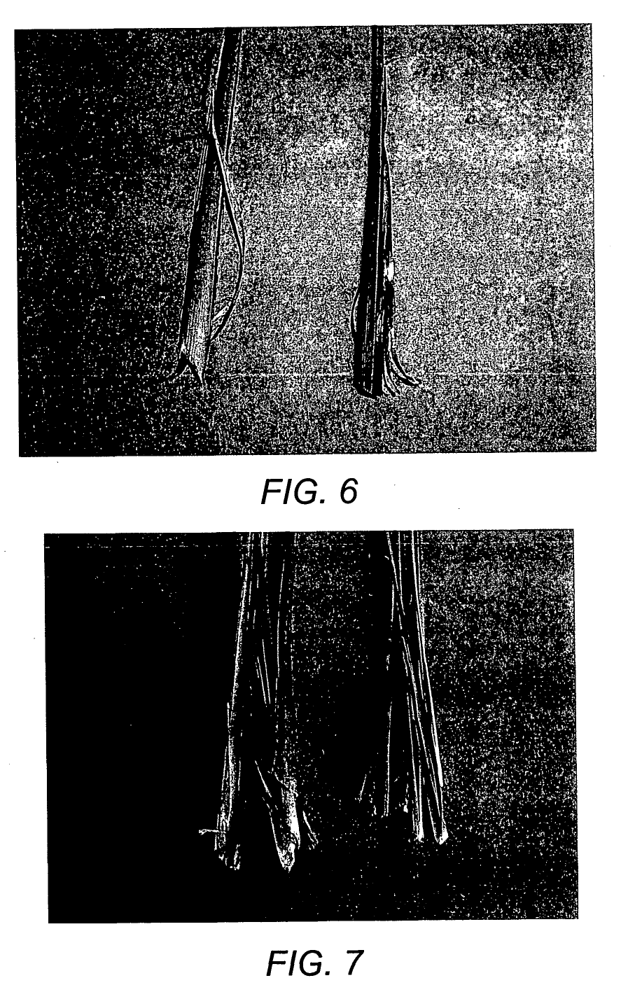

The coloration of ‘LM400’ is distinct from that of ‘LM300’ (FIG. 3 and FIG. 4). ‘LM400’ is a blue/grey color and ‘LM300’ is a green color. Furthermore, ‘LM300’ has purple basal shoots, whilst ‘LM400’ has white-green basal shoots (FIG. 6 and FIG. 7).

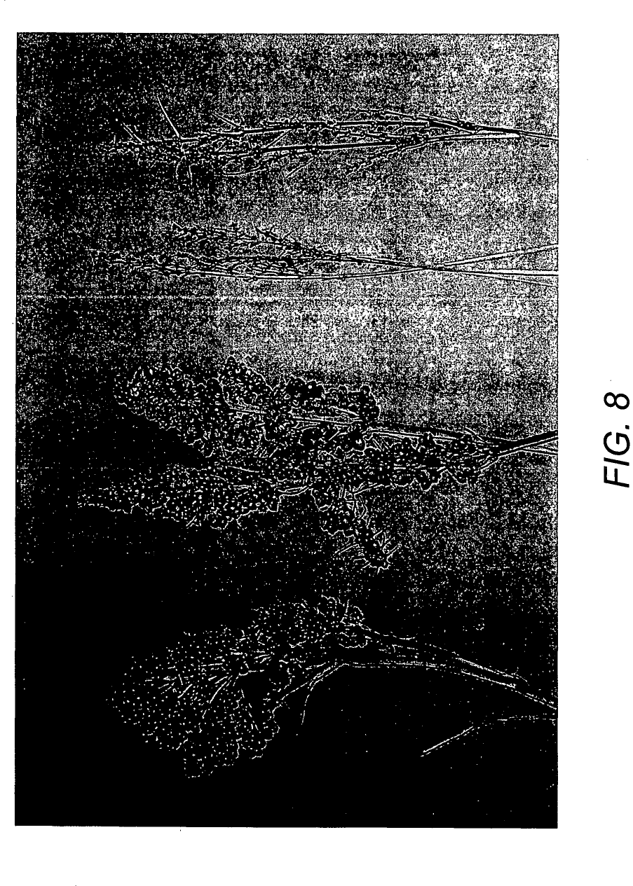

‘LM400’ has a smaller flower spike than ‘Katrina’ and ‘Cassica’ (FIG. 8). ‘LM400’ has a flower spike that is a little shorter than that of ‘LM300’. To date, ‘LM400’ has not produced viable seed, whilst ‘Cassica’ and ‘Katrina’ typically produce viable seed in female plants.

The flower spike of ‘Katrina’ and ‘Cassica’ are relatively spiky to touch, while the flower spike of ‘LM400’ is less spiky. ‘LM400’ is a little spikier than ‘LM300’ to touch.

The novelty and distinctiveness of ‘LM400’ as compared with other varieties of Lomandra longifolia is further illustrated by vegetative (Table 1) and inflorescence (Table 2) measurements.

TABLE 1

| | | Basal | | |
|---|---|---|---|---|
| | Blade | Shoot | Blade | |
| | Width | Membrane | Length* | |
| Variety | (mm) | Color | (mm) | Color |
| ‘LM300’ | 2.5 to 3.5 | Purple/Dark | 557.5 | Green |
| | | Brown | | |
| ‘LM400’ | 3 to 5 | White/Green & | 714 | Blue/Grey |
| | | Light Brown | | |
| ‘Katrina’ | 7 to 10 | Purple | 947.5 | Green |
| ‘Cassica’ | 10 to 15 | White/Green | 1095 | Blue/Grey |
| Common Lomandra^{#} | 10 to 16 | Variable | 1085 | Green- |
| | | | | Blue/Grey |

*Indicates average.
^{#}Not subject to Australian Plant Breeder's Rights.
Test Plots were planted on Jul. 6, 2001 at Abulk Nursery in Windsor, New South Wales, Australia by Abulk Pty Ltd.

TABLE 2

| | Flower Spike | Flower Spike | |
|---|---|---|---|
| Variety | Length* (mm) | Width* (mm) | Spikiness |
| ‘LM300’ | 195 | 25 | Very Little Spikiness |
| ‘LM400’ | 185 | 25 | Medium Spikiness |
| ‘Katrina’ | 240 | 65 | Medium to Very Spiky |
| ‘Cassica’ | 230 | 48 | Very Spiky |
| Common | 240 | 51 | Very Spiky |
| Lomandra^{#} | | | |

*Indicates average.
^{#}Not subject to Australian Plant Breeder's Rights.
Test Plots were planted on Jul. 6, 2001 at Abulk Nursery in Windsor, New South Wales, Australia by Abulk Pty Ltd.

Asexual Reproduction

After its initial discovery, ‘LM400’ was transplanted into a 140 mm pot for further trials and testing. After divisions were made for several subsequent generations, ‘LM400’ was observed to retain color, size, and fine leaf characteristics that were noted in the original ‘LM400’ seedling. ‘LM400’ was then divided into many larger pots for further evaluation. During these divisions, it was noted that ‘LM400’ could produce 5 to 8 divisions per 140 mm pots. In comparison, ‘Katrina’, ‘Cassica’ and common Lomandra longifolia only produced 2 to 5 divisions. More extensive data regarding division rates per 140 mm pot and 300 mm pot of ‘LM400’ as compared with other Lomandra longifolia are presented in Table 3.

TABLE 3

| | Divisions per 140 mm | Divisions per 300 mm |
|---|---|---|
| Variety | Pot (Range) | Pot (Range) |
| ‘LM300’ | 7 to 10 | 24 to 32 |
| ‘LM400’ | 5 to 8 | 22 to 26 |
| ‘Katrina’ | 4 to 5 | 8 to 13 |
| ‘Cassica’ | 2 to 3 | 6 to 7 |
| Common Lomandra* | 2 to 3 | 6 to 9 |

*Not subject to Australian Plant Breeder's Rights.
Test Plots were located at Abulk Nursery in Windsor, New South Wales, Australia. Divided between Jul. 6, 2001 and Dec. 15, 2001.

Divisions of ‘LM400’ root within 3 to 4 weeks, which is faster than the 5 to 7 weeks typical for ‘Cassica’. The survival rate of ‘LM400’ division is 97% whilst ‘Cassica’ is approximately 80%. A more detailed comparison of division survival rate is provided in Table 4.

TABLE 4

| | No. of Divisions that |
|---|---|
| Variety | Survived Out of 200 |
| ‘LM300’ | 198 |
| ‘LM400’ | 194 |
| ‘Katrina’ | 170 |
| ‘Cassica’ | 159 |
| Common Lomandra* | 163 |

*Not subject to Australian Plant Breeder's Rights.
Test Plots were located at Abulk Nursery in Windsor, New South Wales, Australia. Divided between Jul. 6, 2001 and Dec. 15, 2001.
Potting mix used contained 50% sand and 50% peat. Plants were divided into 90 mm × 50 mm × 50 mm tubes.

Environmental Tolerances

‘LM400’ has shown potential for shade tolerance and further shade tolerance tests are underway. The winter hardiness of ‘LM400’ is at least to zone 8a, and evaluation of winter hardiness is ongoing. ‘LM400’ has been observed to hold color to −8 degrees Celsius without any noticeable change in appearance of the plant. With testing at −12 degrees Celsius, minor frost burning was observed at the tips of the leaves, with slight paling in color. ‘LM400’ has excellent drought tolerance compared with other Lomandra longifolia varieties, most likely due to the massive, deep root system. After severe wilting, ‘LM400’ has been noted to recover with watering. ‘LM400’ does well in sandy soils, but also tolerates heavy, clay-type soils well.

Disease Resistance

‘LM400’ has good resistance to root rot compared with most other Lomandra longifolia, but is not as resistant as ‘LM300’.

That which is claimed is:

1. A new and distinct variety of Lomandra longifolia plant named ‘LM400’, substantially as described and illustrated herein.
